(19) United States
(12) Patent Application Publication
(10) Pub. No.: US 2022/0211282 A1
(43) Pub. Date: Jul. 7, 2022

(54) HEMODYNAMIC MONITORING SYSTEM AND METHOD AND HARNESS FOR SAME

(71) Applicant: ENDOTRONIX, INC., Lisle, IL (US)

(72) Inventors: Harry D. Rowland, Plainfield, IL (US); Michael L. Nagy, Lombard, IL (US); Angad Singh, Lisle, IL (US); Balamurugan Sundaram, Peoria, IL (US); Omid Forouzan, Chicago, IL (US); Thomas Wilschke, Chicago, IL (US); Stuart Allan Karten, Marina del Rey, CA (US); Eric William Olson, Marina del Rey, CA (US); Christian Gunnar Denhart, Marina del Rey, CA (US); Cezanne-Simon Van Rensselaer Farris-Gilbert, Marina del Rey, CA (US)

(21) Appl. No.: 17/569,989

(22) Filed: Jan. 6, 2022

Related U.S. Application Data
(60) Provisional application No. 63/134,322, filed on Jan. 6, 2021.

(51) Int. Cl. A61B 5/02; A61B 5/00; A61B 5/08; A61B 5/01; A61B 5/024; A61B 5/021

(52) U.S. Cl. CPC A61B 5/02028; A61B 5/0022; A61B 5/0031; A61B 5/0816; A61B 5/02108; A61B 5/02405; A61B 5/7475; A61B 5/6831; A61B 5/686; A61B 5/01

(57) ABSTRACT

Disclosed is a hemodynamic monitoring system, and method and associated harness device. The method and system are configured to allow a user that has received a permanent sensing implant within their vasculature to place a reader device in a hands free manner in communication with the implant through the chest of the user to measure at least one hemodynamic parameter such as pulmonary artery pressure. The reader device may be worn by the patient with a harness device to be configured to wirelessly communicate with said implant when the patient is in a specific patient state, such as resting, exercising, recovering, seated, or supine. The reader may be configured to take measurements from the implant when the patient is in the specific patient state, in order to acquire data from said implant related to said parameter and to upload said data to an external device.

### CROSS-REFERENCE TO RELATED APPLICATIONS

This application claims priority to and the benefit of U.S. Provisional Patent Application No. 63/134,322 filed Jan. 6, 2021 and titled: “HEMODYNAMIC MONITORING SYSTEM AND METHOD which is hereby incorporated by reference in its entirety.

This application is also related to U.S. Pat. No. 10,206,592 entitled “PRESSURE SENSOR, ANCHOR, DELIVERY SYSTEM AND METHOD as well as U.S. patent application Ser. No. 17/138,081 entitled “ANCHORING SYSTEM FOR A CATHETER DELIVERED DEVICE,” and U.S. Pat. No. 10,993,669 entitled “ANCHORING SYSTEM FOR A CATHETER DELIVERED DEVICE,” filed on Apr. 20, 2018 which are hereby incorporated by reference in its entirety.

This application is also related to U.S. Pat. No. 10,638,955 entitled “PRESSURE SENSING IMPLANT,” filed on Jul. 19, 2016 which is a continuation-in-part of U.S. Pat. No. 10,226,218 entitled “PRESSURE SENSING IMPLANT” filed on Sep. 16, 2015, each of which are incorporated by reference in its entirety. Further, this application is also related to the following U.S. Pat. Nos. 10,430,624; 10,003,862; 9,894,425; 9,489,831; 9,721,463; 9,305,456; and 8,432,265 and to U.S. patent application Ser. No. 16/040,034 each of which is also incorporated by reference in their entirety.

### FIELD OF INVENTION

This application relates to systems, methods and assemblies for measuring hemodynamic parameters in various patient states using wireless implants and wearable or portable devices. In particular, this application is directed to systems comprising wireless implants and wearable devices that measure hemodynamic parameters of a patient.

### BACKGROUND

The practice of cardiology relies heavily on diagnostic measurements of hemodynamic parameters. Examples include cardiac output, stroke volume, pulmonary vascular resistance, pulmonary capillary wedge pressure, ventricular filling pressure, arterial blood pressure, central venous pressure, ejection fraction and pulmonary artery pressure. Some of these measurements can be used as surrogates to estimate others. The measurements are used to diagnose pathologies and prescribe therapy.

In many cases there may be clinical value in obtaining these measurements in different patient states including ambulatory states. Examples of patient states include: at rest, during exercise, after exercise, during sleep, seated, standing, supine, recumbent, limbs elevated, during symptomatic episodes, during or after dialysis related therapies, deep or suspended breathing, Valsalva maneuvers, different times of day, etc. Despite their potential clinical value, many of these measurements are limited, difficult or impractical in many of the patient states, given limitations of the present art. For example, pulmonary artery pressure during exercise (“exercise PAP”) is used to diagnose certain types of pulmonary artery hypertension, pulmonary hypertension, or heart failure. Right heart catheterization is the present state of the art for measuring PAP. A catheter is inserted into the patient's jugular vein, through the two chambers of the right heart, and into the pulmonary artery. The catheter is essentially a liquid tube that communicates pressure from its distal end to a transducer outside the body. Catheters with wired in-body microsensors also exist. The patient then walks on a treadmill or sits or lies supine while pedaling a stationary bicycle while pressure measurements are taken.

This methodology brings about disadvantages. They include risks to the patient such as discomfort or pain; adverse events such as bleeding from the access site, thrombus, reaction to sedation, infection, and physical stress on the vasculature. They further include nonuniformity in testing due to variations in exercise equipment, protocols, patient postures, errors in sensor leveling, etc. Further, patients may be prone to “white coat syndrome” in which the stress of being in a clinic environment with a catheter in one's jugular vein causes physiological parameters to change from typical values in the daily environment. Equipment and staff time are costly, and appointments may require long wait times. Travel to a clinic with the proper equipment can be physically or economically difficult for unhealthy, remotely located, or lower income patients. Patients mental or physical ability or willingness to achieve peak exercise levels may be reduced by discomfort from the catheter. Infrequent measurements increase the risk of conclusions being drawn based on statistical outliers. And finally, right heart catheterizations performed by fluid-filled catheters can lose accuracy due to fluid column weight, caused by improper leveling of the sensor with the tube end. Proper leveling is complicated when the catheter system is in motion during exercise.

Noninvasive means may be an improvement over invasive ones for measuring hemodynamics in different patient states. Continuing with our example of exercise PAP measurement, wireless implant devices for measuring PAP such as Abbott Medical's CardioMEMS HF System may offer an improvement over the invasive catheter method described above. One version of the CardioMEMS HF System is used in the patient's home and includes a large, stationary external reader that plugs into a wall outlet on which the patient must lie supine during measurements. Small changes in the patient's body position can cause inaccurate readings, making it incompatible with dynamic exercise. An in-clinic version of the CardioMEMS HF System includes a reader device that couples a handheld antenna to a large base station via a thick cable. While an improvement over the home version, the reader device is not fully handheld and patient motion is limited by the cable. Another example of noninvasive means for measuring hemodynamics is the V-LAP System by Vectorious Medical. The V-LAP includes a reader device and implant configured to measure Left Atrial Pressure (LAP), another hemodynamic parameter. While smaller than CardioMEMS HF System reader and battery powered, the V-LAP reader device requires the patient to encircle the thorax with a large, sash-like antenna strap, any movement of which during the reading may cause unacceptable inaccuracy.

In view of these issues with the prior art, it is desirable to incorporate a system with small, portable, easily wearable reader devices that allow fast and simple wireless communication with permanently implanted sensors that accurately measure hemodynamic parameters. They should be operable quickly and efficiently by minimally trained clinical personnel, non-clinical users, or the patients themselves, in a variety of patient states and environments. The devices should be capable of stable, accurate measurement during the typical body movements and postures assumed in states such as exercise, sleep, ad hoc symptomatic episodes or physician requests, daily life, etc., and in environments that include cardiology/pulmonology/nephrology clinics, the home, outdoors, hospitals, physical therapy facilities, infusion centers and other medical facilities. Incorporation of activity monitoring sensors and algorithms to detect and classify different activities may also be advantageous to correlate the activity with the measured hemodynamic parameters and derive additional insights.

### SUMMARY OF THE DISCLOSURE

In one embodiment, provided is a hemodynamic monitoring method that may include the following steps: implanting a patient with a wireless sensing device that measures at least one hemodynamic parameter, such as pulmonary artery pressure; providing the patient with a portable reader device that can be worn by the patient and configured to wirelessly communicate with said wireless sensing device when the patient is in a specific patient state, such as resting, exercising, recovering, seated, or supine. The reader is operated to take measurements from the implant when the patient is in a specific patient state in order to acquire data from said wireless sensing device related to at least one hemodynamic parameter and uploading said data to an external device. The implant may be placed in the cardiovascular system and the measured hemodynamic parameter may be pulmonary artery pressure. The activity information of the patient may be acquired from activity monitoring sensors and associated algorithms, or may be provided manually by the patient or other reader operator such as a caregiver. A clinician may be provided with a platform to prescribe or modify exercise protocols and to use exercise-based measurements for treatment decisions. The clinician may also be provided with a platform to monitor the acquired and uploaded data during exercise testing. The reader device may also be placed in communication with another electronic or medical device that monitors activity (e.g. accelerometer, heartrate, GPS, etc.) to signal when to take a reading or to stop taking readings.

In another embodiment, provided is a hemodynamic monitoring system comprising a wireless implantable sensor that measures a hemodynamic parameter and a wireless reader that communicates with said implanted sensor. The reader has a small, portable form factor and is battery powered. The reader may be hand-held and self-operated by a patient having the implanted sensor. A wearable harness is configured to securely position the reader to the implanted patient's body such that its position relative to the sensor will be maintained during different patient states and the operation of the reader may be hands-free. This may include the reader responding to patient's verbal commands such as “start reading”, “stop reading” etc. Alternatively, the reader may be controlled via a smartphone or tablet application that is communicatively coupled to the reader wherein such communicative coupling is preferably wireless. The implantable sensor may be placed within a cardiovascular system and configured to measure hemodynamic parameters. The wearable configuration may securely position the external reader to the patient in proximity to the implantable sensor but may also be adjustable to fit a range of body sizes and adapt to a range of optimal reading locations. For some wireless sensor/reader systems, the optimal location may provide the shortest physical link distance between the sensor and reader antennas, or may orient the antennas' relative angles such that maximum energy is coupled. In one embodiment, the reader or an upstream device in communication with the reader may include an exercise mode that takes readings at defined intervals or is linked to an external activity monitoring device to take a reading during the patient's performance of an exercise. Such readings may be triggered by on-board reader sensors such as an accelerometer, indicating start/stop of exercise. In another embodiment, the reader or an upstream device includes a sleep mode that takes readings at defined intervals or is configured to take continuous readings of the implant in the patient during sleep, or long term applications. The device user may directly indicate the patient state to the reader with a control (e.g. pushbutton or touchscreen) or an audible command. The reader or upstream device may contain an algorithm that determines patient state based on measured parameters. For example, an accelerometer indicating a recumbent position plus lowered heart and respiratory rates could indicate sleep; or an accelerometer indicating steps taken in an upright position, with increased heart and respiration rates could indicate exercise.

In another embodiment, provided are embodiments of a harness device for securely placing a wireless reader to a target area on a patient's chest for allowing a reader to communicate with an implant within the patient's body. The harness device comprises a front panel configured to support a reader device along the target area, a shoulder portion placed over a shoulder of the patient or user and attached to the front panel, and a rear portion that extends from the shoulder portion. The front panel may support the reader by a support window placed in or on the front panel having a complimentary shape of an upper portion of the housing of the reader. Alternatively, the support window may be formed by a frame member. The housing of the reader is configured to be placed within the support window such that an antenna of the reader is placed against the target area and aligned with an implant location while an opposing portion of the housing opposite the base extends through and is supported by the support window. The reader and harness may be configured to allow quick and easy reader insertion into or removal from the harness. In this way, the same reader device can be used as a handheld, hands-free with the harness, or placed in a charging station or dock.

### BRIEF DESCRIPTION OF THE DRAWINGS

Objects and advantages together with the operation of the invention may be better understood by reference to the following detailed description taken in connection with the following illustrations, wherein:

FIG. 1 illustrates a block diagram of a prior art passive wireless implant device and reader system according to U.S. Pat. No. 9,305,456;

FIG. 2 illustrates an embodiment of a prior art wireless implant device;

FIG. 3 illustrates an embodiment of a reader device and a docking station according to the prior art;

FIG. 4 illustrates an embodiment of a patient using the reader device to take a wireless reading from an implant device implanted within the pulmonary artery of the patient according to the prior art;

FIG. 5 illustrates a schematic diagram of a target area for a user to place a reader device to communicate with an implant placed within the cardiovasculature of the patient according to the present disclosure;

FIG. 6A is a perspective view of an embodiment of a harness device for supporting a reader device at a target area on a user for communicating with the implant according to the present disclosure;

FIG. 6B is a perspective side view of the harness device of FIG. 6A;

FIG. 6C is a perspective rear view of the harness device of FIG. 6A;

FIG. 6D is a perspective view of an underside of the harness device of FIG. 6A;

FIG. 6E is an enlarged view of the harness device of FIG. 6A;

FIG. 6F are various views illustrating the adjustability of the harness device of FIG. 6A;

FIG. 7A is a perspective view of another embodiment of a harness device for supporting a reader device at a target area on a user for communicating with the implant according to the present disclosure;

FIG. 7B is a perspective rear view of the harness device of FIG. 7A;

FIG. 7C is a view of the reader device separate from the harness device of FIG. 7A;

FIG. 7D is a perspective view of the top portion of the harness device of FIG. 7A;

FIG. 7E is a perspective view of the harness device of FIG. 7A;

FIG. 7F is a perspective front view illustrating the adjustability of the harness device of FIG. 7A;

FIG. 7G is a perspective rear view illustrating the adjustability of the harness device of FIG. 7A;

FIG. 8A is a perspective view of another embodiment of a harness device for supporting a reader device at a target area on a user for communicating with the implant according to the present disclosure;

FIG. 8B is a perspective rear view of the harness device of FIG. 8A;

FIG. 8C is a view of the reader device separate from the harness device of FIG. 8A;

FIG. 8D is a perspective view of the reader device in phantom with the harness device of FIG. 8A;

FIG. 8E is a perspective front view illustrating the adjustability of the reader device relative to the harness device of FIG. 8A;

FIG. 8F is a perspective bottom view illustrating the adjustability of the harness device of FIG. 8A;

FIG. 8G is a perspective side view of another embodiment of the harness device of FIG. 8A;

FIG. 9 illustrates an embodiment of a harness device with a reader device attached to a user in an upright position and in communication with a hemodynamic monitoring system;

FIG. 10 illustrates another embodiment of a harness device with a reader device attached to a user while in the upright position;

FIG. 11 illustrates a schematic diagram of an embodiment of a hemodynamic monitoring system including a main module and an in situ module according to one embodiment of the present disclosure;

FIG. 12 illustrates screen shots of a graphical user interface of a user facing display of the hemodynamic monitoring system according to the present disclosure;

FIG. 13 illustrates a seated PA pressure graph and a heart rate graph indicated user data tracked by the hemodynamic monitoring system according to the present disclosure;

FIG. 14A illustrates a “baseline” pressure graph tracked by the hemodynamic monitoring system according to the present disclosure;

FIG. 14B illustrates a “walk” pressure graph tracked by the hemodynamic monitoring system according to the present disclosure;

FIG. 14C illustrates a “recovery” pressure graph tracked by the hemodynamic monitoring system according to the present disclosure;

FIG. 15 is a graph that illustrates various data readings tracked by the hemodynamic monitoring system;

FIG. 16 is an embodiment of an interactive graph that illustrates various data readings are tracked by the hemodynamic monitoring system as a user is in various patient states; and

FIG. 17 illustrates a waveform graph window expanded from the interactive graph of FIG. 16

### DETAILED DESCRIPTION

Reference will now be made in detail to exemplary embodiments, examples of which are illustrated in the accompanying drawings. It is to be understood that other embodiments may be utilized and structural and functional changes may be made. Moreover, features of the various embodiments may be combined or altered. As such, the following description is presented by way of illustration only and should not limit in any way the various alternatives and modifications that may be made to the illustrated embodiments.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

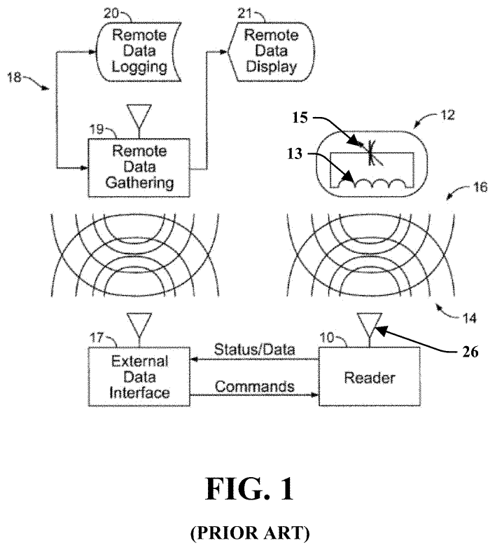

FIG. 1 illustrates an example of applicant's passive wireless sensor system that includes a reader 10 in remote communication with a sensor 12 as disclosed by U.S. Pat. No. 9,305,456. The reader 10 is capable of exciting the sensor 12 by transmitting a signal, such as a radio frequency (“RF”) pulse, at or near the resonant frequency of the sensor 12. The sensor 12 may emit a ring frequency for a short period of time in response to the excitation pulse from the reader 10. The sensor 12 may be a passive device, capable of emitting a ring signal in response to an excitation signal at or near the resonant frequency of the sensor 12. The sensor 12 may be configured to sense a specific hemodynamic parameter. For example, the sensor 12 may include a fixed inductor 13 and a capacitor 15 that varies based on the sensed parameter. The varying capacitance alters the resonant and ring frequencies of the sensor 12. The corresponding reader 10 may employ corresponding signals to activate the sensor 12.

The reader 10 may excite the sensor 12 by transmitting an excitation pulse 14 in the vicinity of the sensor 12. For example, the reader may emit a radio frequency (“RF”) excitation pulse 14 at or near the resonant frequency of the sensor 12. The sensor 12 may emit a ring signal 16 in response to the excitation pulse 14. The reader 10 may determine the frequency of the ring signal 16 in order to determine the sensed parameter value. The reader 10 may also communicate with a data interface 17. The reader 10 and data interface 17 may be connected directly or indirectly, or may communicate via a remote connection. The reader 10 may send information, such as data related to the sensor 12, to the data interface 17. The reader 10 may further send information regarding the status of the reader 10 to the data interface 17. The data interface 17 may communicate with a remote data system 18 to exchange status and control signals, as well as provide sensor data. The remote data system 18 may include a data gathering module 19 to receive data from the data interface 17, a data logging module 20 to store the received data, and a data display 21 to display the sensor data.

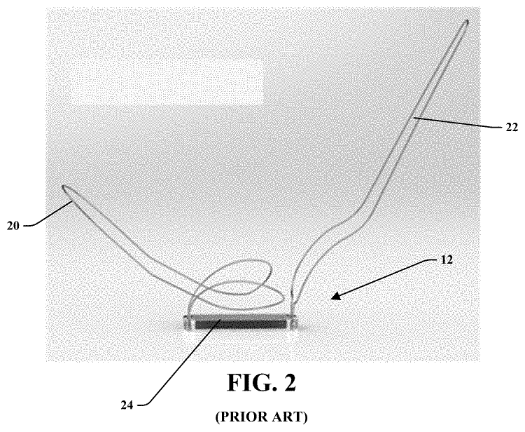

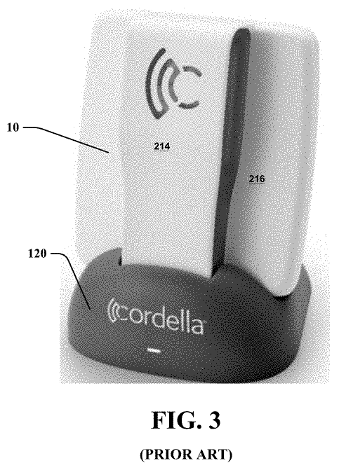

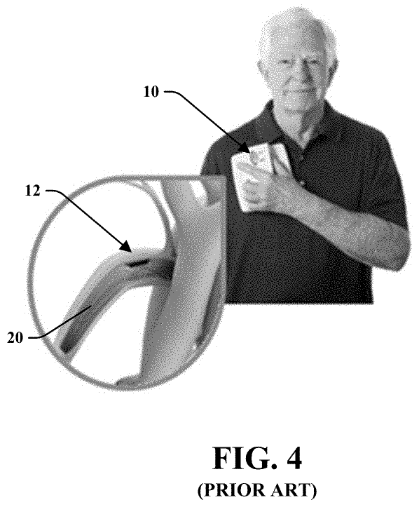

FIG. 2 illustrates an embodiment of an implant 12 having proximal anchor 20 and a distal anchor 22 that is configured to be implanted into the pulmonary artery of a user. The implant 12 may include a housing 24 and is configured to measure a hemodynamic parameter, such as pulmonary artery pressure, as disclosed by at least applicant's U.S. Pat. No. 10,638,955 which is incorporated herein in its entirety. FIG. 3 illustrates an embodiment of a reader device 10 placed in a docking station 120. The reader 10 may be placed in communication with the implant 12 by a user that has been implanted with the implant 10 as illustrated by FIG. 4. In one embodiment, the reader may include an enlarged base portion 216 and an upper portion 216 configured to allow the user to grasp and hold the reader relative to the chest of the patient. The reader may have a weight of about 2.5 pounds. The docking station 120 may allow the reader to charge or otherwise be placed in communication with a network infrastructure to communicate data and commands between a remote location and the reader 10 which is desired to be with the patient/user. An example of a reader 10 and docking station 120 in communication with a network system is disclosed by commonly owned U.S. Pat. No. 10,430,624 which is incorporated herein in its entirety.

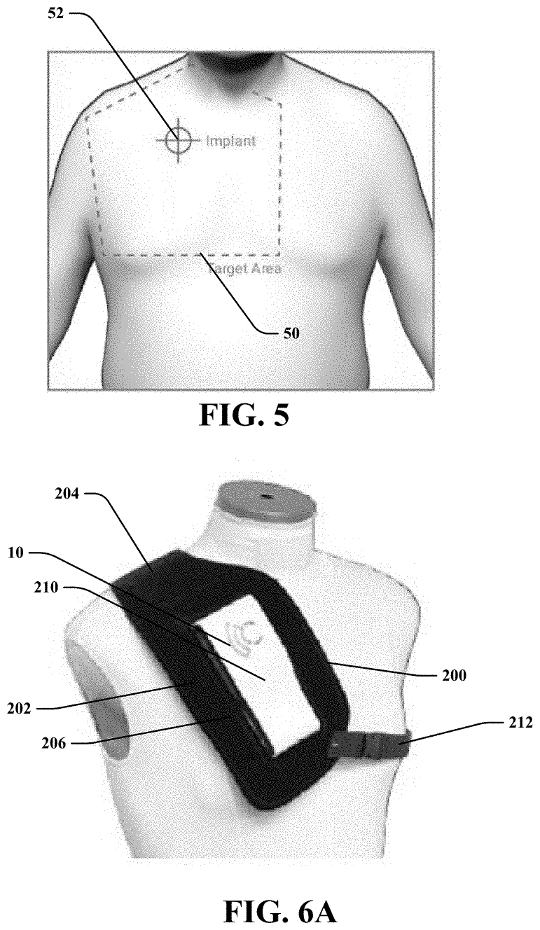

FIG. 5 illustrates a schematic diagram of a target area 50 for a user to place the reader 10 to properly and accurately communicate with an implant 12 placed within the cardio vasculature of the patient according to the present disclosure. The target area 50 is generally along the upper front chest area of an implant recipient. The implant 12 is preferably placed within the pulmonary artery to take readings of hemodynamic parameters, such as pulmonary artery pressure (“PAP”). However, the location of the implant 12 is generally positioned close to location 52 within the target area 50 wherein the distance and proximity of the reader 10 relative to the implant location 52 and implant 12 thereunder impacts signal communication strength. It is desirable to have close directional alignment and angular alignment of the antenna 26 of the reader 10 with the inductor 13 of the implant 12 to accurately communicate the pulse and ring signals therebetween.

The instant disclosure is related to a hemodynamic monitoring system and method and harness device for same. The hemodynamic monitoring system incorporates the reader 10 and implant 12 as well as the back end network and software infrastructure used in a particular manner as disclosed herein. The harness device 200 allows a user to place the reader 10 in close alignment with the implant 12 to take readings that can be communicated to the reader 10 and then communicated to the network for measuring, tracking, recording, diagnosing, or creating a profile of the user's measured hemodynamic parameters. Various embodiments of the harness device 200 are disclosed herein and allow for modified usage of known reader and implant devices to improve the usability of the hemodynamic monitoring system. In one embodiment, the harness device in addition to the reader, implant, and backend operating system can be used to obtain pulmonary artery pressure measurements on patients while they are performing a defined exercise routine. This may provide a unique ability to obtain these measurements without the limitations of a catheter and outside a clinical environment, in a hands free configuration to allow user movement is a desirable feature. Further, the disclosed harness devices may be adjustable and conducive to fitting various sized body types that allow the wearer to move the reader into the ideal position relative to their implant location, at which time the harness will provide a secure means of holding the reader on the chest during exercise or other patient states.

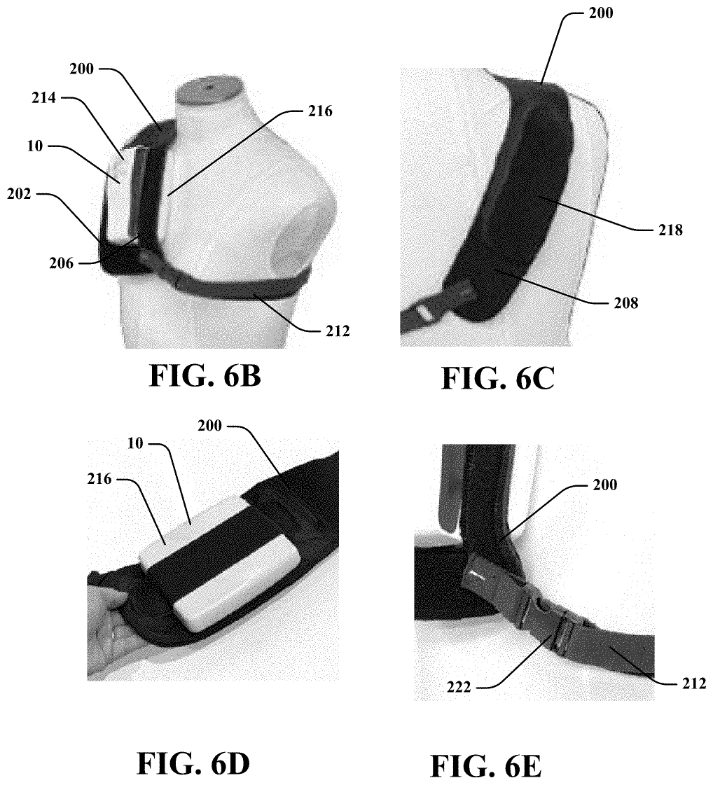

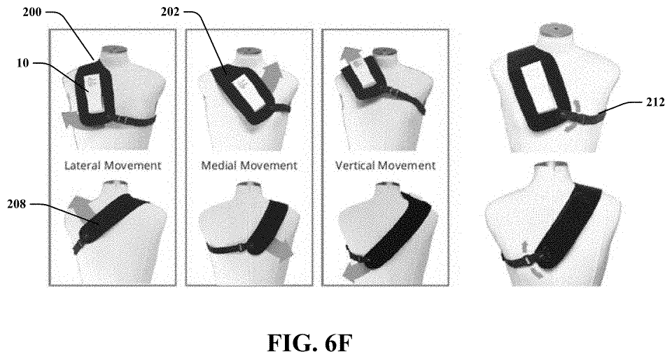

FIG. 6A illustrates an embodiment of the harness device 200 for supporting a reader 10 at a target area 50 on a user for communicating with the implant 12 according to the present disclosure. The harness device 200 may be made of a flexible but sturdy materials or any combination of materials such as nylon, neoprene, synthetic fabrics, leather, cotton, wool, spandex, gore-tex, bamboo, polymer, polyester, polypropylene, rubber, Teflon, or any combination of such materials to form a lightweight, possibly breathable and comfortable fit with a user. In one embodiment, no metal material is used in the harness. The harness 200 may be configured to fit a specific size person or have the potential to fit a wide range of patients that is adjustable. It is configured to hold the reader securely with as little relative movement as possible from its desired location in proximity with the implant 12 at the implant location 52 in the target area 50.

The harness 200 can be placed directly on the skin of a patient or over clothing of a patient. In one embodiment, the harness device includes a front panel 202 configured to support the reader device 10 along the target area 50, a shoulder portion 204 placed over a shoulder of the user and attached to the front panel 202, and a rear portion 208 that extends from the shoulder portion 204.

FIG. 6B is a perspective side view of the harness device 200 and illustrates that in one embodiment, the front panel 202 supports the reader 10 by a support window 206 placed in the front panel 202 having a complimentary shape of an upper portion 214 of the housing 210 of the reader 10. The housing 210 is configured to be placed within the support window 206 such that an antenna 26 of the reader 10 within a base portion 216 of the housing 210 may be placed against the target area 50 and aligned with the implant location 52 while an opposing portion of the housing 210 opposite the base extends through and is supported by the support window 206. In this embodiment, the base portion 216 of the reader housing 210 has an enlarged perimeter shape relative to the upper portion 214.

FIG. 6C is a perspective rear view of the harness 200 and illustrates the rear portion 208 that extends from the shoulder portion 204. A counterweight 218 may be placed in the rear portion 208 to assist with balancing the harness 200 on the body of a user. The counterweight 218 may be any size and in one embodiment is about 0.5 lbs. FIG. 6D illustrates the underside of the harness 200 and includes a strap 220 to secure the base portion 216 of the reader housing 210 while the upper portion 214 of the reader housing 210 extends through the support window 206. The strap 220 may be made of an elastic material for ease of inserting and removing the reader 10 from the harness. FIG. 6E illustrates a strap that selectively attaches the front panel 202 to the back portion 208. The strap 212 may include a buckle 222 to adjust the length of the strap and allow it to be easily detached or attached. The strap 212 may be configured to extend from the back portion 208, under a user's arm, to the front panel 202.

FIG. 6F illustrates various views illustrating the adjustability of the harness device of FIG. 6A. The front panel is configured for lateral, medial and vertical movement while the strap 212 may be rotatable relative to the front panel 202 and back portion 208.

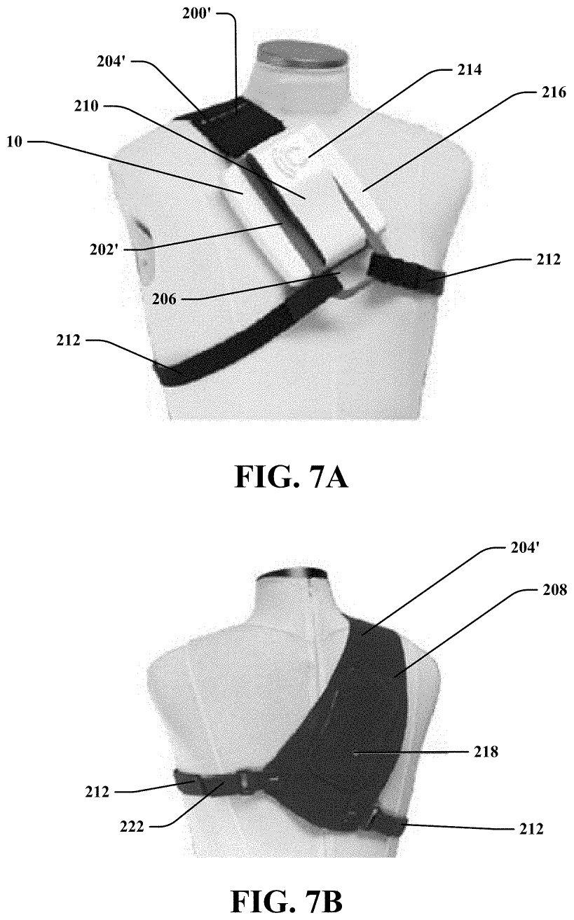

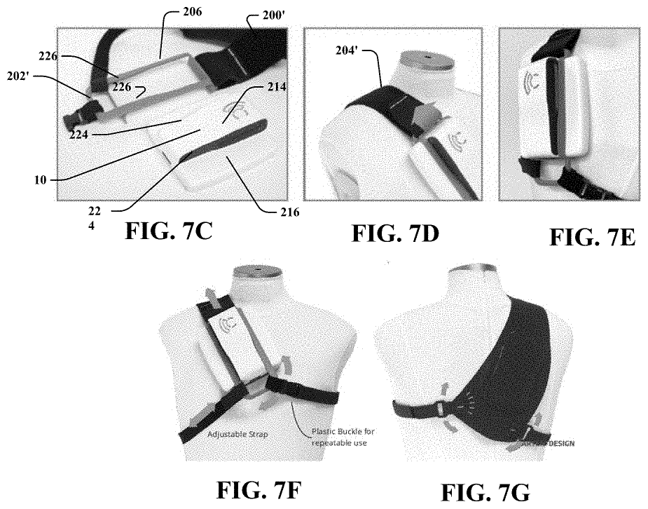

FIG. 7A is a perspective view of another embodiment of a harness device 200′ for supporting a reader device at a target area 50 on a user for communicating with the implant according to the present disclosure. The harness 200′ includes a front panel 202′ configured to support the reader device 10 along the target area 50, a shoulder portion 204′ placed over a shoulder of the user and attached to the front panel 202′, and a rear portion 208 that extends from the shoulder portion 204′. In this embodiment, the front panel 202′ is a rigid frame member having a support window 206 configured to receive the upper portion 214 of the reader housing 10 to support it therein. The support window 206 is placed in the front panel 202 having a complimentary shape of the upper portion 214 of the housing 210 of the reader 10. The housing 210 is configured to be placed within the support window 206 such that the antenna 26 of the reader 10 within the base portion 216 of the housing 210 may be placed against the target area 50 and aligned with the implant location 52 while an opposing upper portion 214 of the housing 210 opposite the base extends through and is supported by the support window 206.

FIG. 7B is a rear view of the harness device 200′ and illustrates the rear portion 208 that extends from the shoulder portion 204′. A counterweight 218 may be placed in the rear portion 208 to assist with balancing the harness 200′ on the body of a user. The counterweight 218 may be any size and in one embodiment is about 0.5 lbs. FIG. 7B illustrates a first strap 212 and a second strap 212 that may selectively attach the front panel 202′ to the back portion 208. The straps 212 may include a buckle, hook and loop fasteners or other type of strap attachment 222 to adjust the length of the strap and allow it to be easily detached or attached therefrom. The straps 212 may be configured to extend from the back portion 208, under a user's arms, to the front panel 202.

FIG. 7C illustrates the reader 10 separate from the front panel 202′. The rigid frame of the front panel 202′ defines the support window 206 that includes space to allow the straps 212 to connect thereto. The rigid frame of the front panel 202′ may have contoured shapes that are complementary to the base portion 216 and upper portion 214 of the reader housing 210. In one such embodiment, the upper portion 214 of the reader housing 210 may include slightly tapered sides 224 wherein the front panel 202′ includes frame members 226 having a complementary tapered shape to the tapered sides 224 of the upper portion 214 of the reader housing 210. This configuration allows for the reader 10 to easily slide into the front panel 202′ and be sufficiently supported within the support window 206 of the harness 200.

FIG. 7D illustrates how the shoulder portion 204′ may be attached to the front panel 202′ through a space and include hook and loop fasteners to allow for adjustability. FIG. 7E illustrates the reader 10 placed within the front panel 202′ of the harness 200′ and FIGS. 7F and 7G illustrate the adjustability of the straps 212 and buckles 222 relative to the various components of the harness 200′.

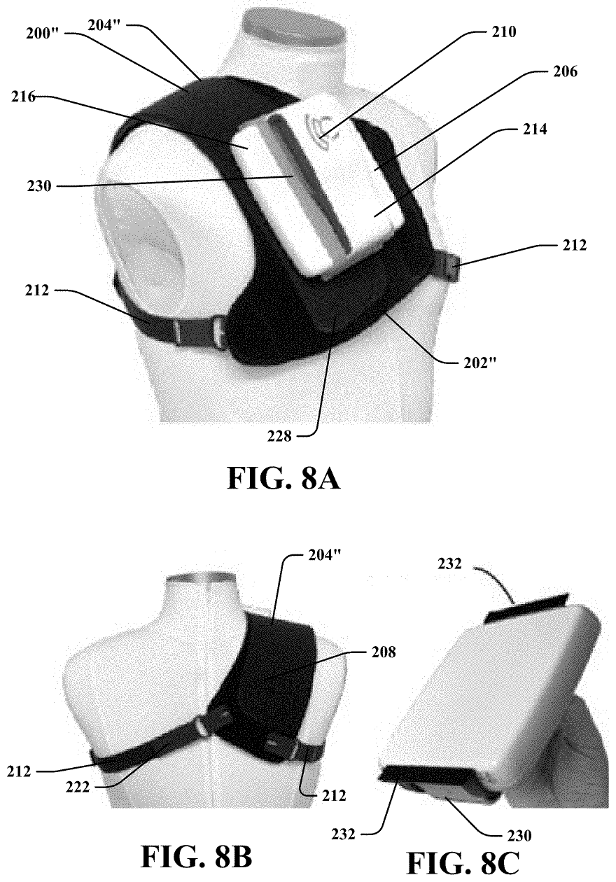

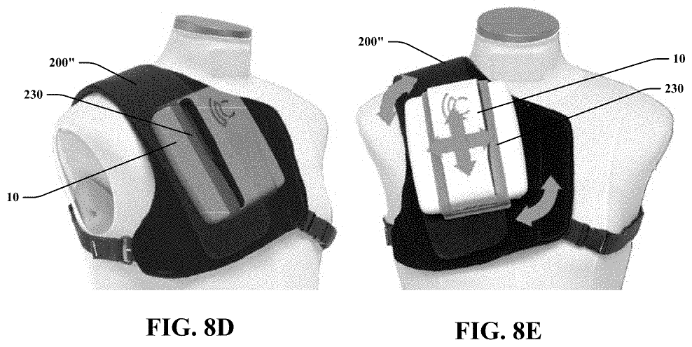

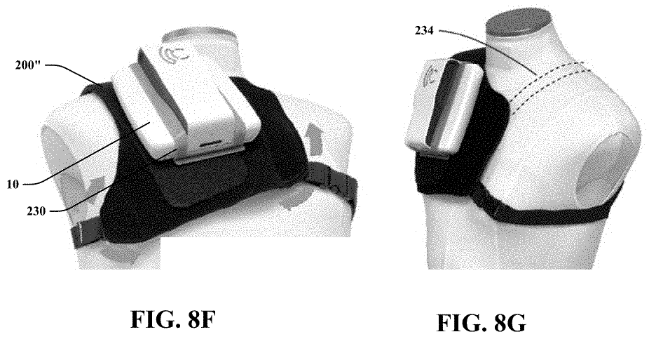

FIG. 8A is a perspective view of another embodiment of a harness device 200″ for supporting a reader device at a target area 50 on a user for communicating with the implant according to the present disclosure. The harness 200″ includes a front panel 202″ configured to support the reader device 10 along the target area 50, a shoulder portion 204″ placed over a shoulder of the user and attached to the front panel 202″, and a rear portion 208 that extends from the shoulder portion 204″. In this embodiment, the front panel 202″ includes a hook and loop fastener surface 228 wherein a frame member 230 having a support window 206 is configured to encircle the upper portion 214 of the reader housing 210 to support it therein. The frame member 230 includes tabs 232 extending from the perimeter of the reader housing 210 that include hook and loop fasteners that are configured to be attached to the hook and loop fastener surface 228 on the front panel 202″. The support window 206 of the frame member 230 may include a complimentary shape to the upper portion 214 of the housing 210 of the reader 10. The housing 210 is configured to be placed within the support window 206 such that the antenna 26 of the reader 10 within the base portion 216 of the housing 210 may be placed against the target area 50 and aligned with the implant location 52 while the upper portion 214 of the housing 210 opposite the base extends through and is supported by the support window 206.

FIG. 8B is a rear view of the harness device 200″ and illustrates the rear portion 208 that extends from the shoulder portion 204″. The hook and loop fastener surface 228 may extend from the front panel 202″ over the shoulder portion 204″ and to the rear portion 208. A first strap 212 and a second strap 212 may selectively attach the front panel 202″ to the back portion 208. The straps 212 may include a buckle or hook and loop fasteners or other type of strap attachment 222 to adjust the length of the strap and allow it to be easily detached or attached. The straps 212 may be configured to extend from the back portion 208, under a user's arms, to the front panel 202″.

FIG. 8C illustrates the reader housing 210 separate from the front panel 202″. The frame member 230 that defines the support window 206 includes tabs 232 projecting from opposing sides and extending from the perimeter of the base portion 216 of the reader housing 210. The tabs 232 include hook and loop surface and are configured to be attached and detached and adjusted relative to the fastener surface 228 as illustrated by FIG. 8E. The frame member 230 may have a contoured shape that is complementary to the base portion 216 and upper portion 214 of the reader housing 210. In one such embodiment, the upper portion 214 may include slightly tapered sides 224 wherein the frame member 230 includes a complementary tapered shape to the tapered sides 224 of the upper portion 214 of the reader housing 210. This configuration allows for the reader to easily slide into the frame member 230 and be sufficiently supported within the support window 206 of the harness 200. FIGS. 8D, 8E, and 8F illustrate how the frame member 230 attaches the reader 20 to the harness 200″ and how the reader 10 and straps 212 may be adjustable relative to the user. FIG. 8G illustrates that an over the shoulder strap 234 may optionally be available for any version of the harness.

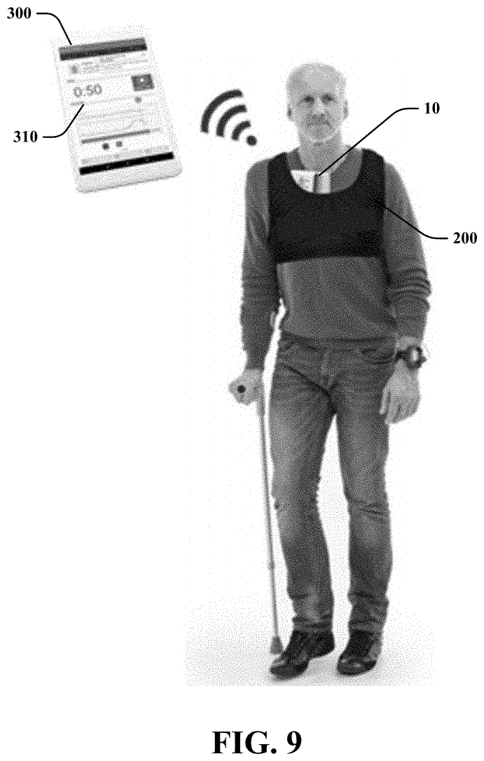

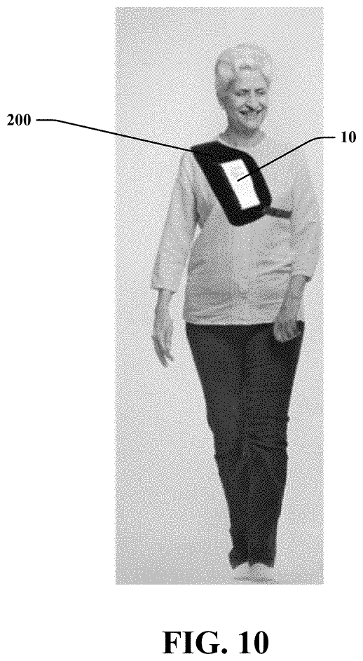

FIGS. 9 and 10 illustrate embodiments of the harness device 200, 200′, 200″ with a reader device 10 attached to a user in an upright position. FIG. 9 illustrates the reader device 10 in communication with a computing device such as a cell phone or tablet. Notably, the hemodynamic monitoring system and method of the disclosure can function in a variety of manners while being placed in the harness device. It allows for the reader 10 to communicate with the implant 12 while the patient is in a variety of patient states. The disclosed embodiments of the harness device may be allowed to provide minor or subtle shifting of the reader along the surface of the chest but restricts angular movement or “roll” relative to the chest plane that would create an angular offset with alignment of the reader antenna 26 relative to the inductor 13 of the implant 12 and target location 52. A secure, close fit is desired for maintaining signal strength between the reader and the implant. The readings taken by the implant in these patient states may be communicated to the reader device to provide valuable information relating to the measured hemodynamic parameters, such as PAP, of a user. The following passages describe various functions and combinations of functions and components that the wearable reader device, in continuous communication with the implant, can perform and any of these combinations are contemplated herein.

The hemodynamic monitoring system may be configured to take one or more readings before, during or after a variety of patient states. These patient states may include the time a patient is exercising and/or performing activities, sleeping, feeling symptomatic (e.g. arrhythmia, dyspnea, chest pain, numbness, palpitations, etc.) or showing measurable clinical signs and/or symptoms (e.g. low pulse oxidation, heart rate, etc). A patient can also be attached to a life-supporting or other medical machines when used to measure hemodynamic and other parameters. These other machines include but are not limited to: dialysis machines, extracorporeal membrane oxygenation (ECMO) machine, blood transfusion devices, ventilators, oxygen cannula, chemotherapy assemblies, external pacemakers, nasogastric or orogastric tube, neural electrodes, epidural or other drip, pic/art lines, chest tube, ECG, etc. A patient state can include any situation where it is advantageous to have a wearable reader such as situations that make it difficult to bring the patient into contact with a large or cumbersome reader devices. Examples of different patient states include but are not limited to: patient performing activities, unconscious, comatose, obese, frail, immobile, mentally impaired, paralytic, palsied, restrained, sedated, undergoing surgery, or in cases where movement causes pain or difficulty (muscular diseases, arthritis, atrophy, burns or skin irritation, etc). The portable or handheld reader device, with or without the harness is well suited for such cases.

In the various embodiments described herein, the harness may be any garment (tight fitting vest, strap, holster, girdle, shirt, adhesive) that is suitable to allow a reader device to be attached to a user while the user exercises, sleeps, or otherwise moves while also taking readings from the permanently implanted implant in the user's cardiovasculature. There may be various configurations of the reader device 10 to allow it to function while it is in constant state of communication with the implant while allowing the user to move. Notably, the reader device can include manual activation to activate or trigger the reader to take a reading, for example by sending a pulse to the implant and receive a ring signal therefrom. Such manual activation could include a pushbutton on the reader device or a remote computer control, activating the reader device via hand or arm gesture, activating the reader device via voice recognition, or activating the reader device by recognizing the fingerprint or face scan of a user. Each of these activations could be to start or stop an ad hoc reading. Alternatively, the readings can be timed readings or manual start/stop which can be programmed through the network or through the user's facing software interface.

In a further embodiment, the hemodynamic monitoring system may include an exercise mode where readings can be triggered or stopped manually as desired during exercise. A sleep mode where readings can be taken continually or intermittently during sleep or daily life. Further, there can be a periodic mode for the reader to take periodic short (e.g. ˜20 seconds) readings, at various intervals such as every few hours throughout the day. Further, the system may include a programmed mode where readings are taken at desired programmed times of day.

In another embodiment, the reader device 10 may communicate (e.g. wire, Bluetooth, MICS) with another medical device (e.g. pulseox, implanted pacemaker, ECG, CPAP machine, etc.) or electronic device (e.g., cell phone, smart watch, tablet, computer) and be configured to take a reading on command from the other such device. For example: a CPAP machine may be configured to communicate with the reader 10 on a sleeping patient to take a reading to record PAP when an apnea event occurs. Further still, the hemodynamic monitoring system may be programmed to analyze the data taken from the reader and command another device (e.g., pulseO2, implanted pacemaker, ECG, CPAP machine, etc.) to take a reading or perform an action when certain criteria are met. The central hub of the hemodynamic monitoring system may be programmed to orchestrate the reader device as well as third party medical devices or electronic devices to program the triggers and thresholds which may all be programmable by user or a clinician.

The reader device may use a built-in accelerometer or GPS to measure steps, distance walked, speed of walk (min/max/avg.), etc. Further, the reader may include exercise tracking information to provide a map of existing or traveled paths overlaid to a geographic map and may communicate this data to the central hub or docking station or display it on a display of a connected or associated electronic device.

The reader device may also be configured to communicate with other off-the-shelf activity monitoring devices such as Fitbit, Apple Watch, or other electronic wearable devices and may include other built-in combination of devices including an ECG, microphone/stethoscope for heart or lung sounds. Further, the reader device may include a built-in microphone or a recorder so the user or patient can record comments when taking readings or be able to receive verbal instructions. For example, when experiencing a symptom such as chest pain, the user may speak “I am having chest pain”. Such a recording may be communicated through the server to the central hub of the system, which could alert caregivers or provide instructions to the patient according to an algorithm. In another embodiment, the reader responds to voice commands from the user such as “start” or “stop”, etc. The reader device may be configured to record date, time, location, ambient pressure and temperature when readings of the implant are performed. The reader may also act as a hub for other devices the patient has (e.g. Bluetooth with pulseO2, BP cuff, ECG, weight scale). Further, the reader may communicate with the user's home or cell phone network to alert a clinician, family member, or emergency providers when certain conditions are detected such as an adverse reading or continual adverse readings. The reader may be programmed to include alarms/alerts for patients when certain conditions occur.

In further embodiments of the hemodynamic monitoring system, the reader device may be configured to allow all backend functions such as data upload, data storage and data processing, to be performed on a processor and database that exists on the docking station or the network or central hub. This may allow the housing of the reader device to be small and lightweight for portable functions. The reader may include an application specific integrated circuit (ASIC) for maximum portability during exercise or movement. The reader device and or the sensor device may use energy harvesting during a user's exercise, motion, body temperature, ionic energy. Further, the reader device may be built into a smart phone or smart watch.

The reader device may be configured to give visual, haptic, or audible commands to a patient that signals to the patient to perform home exercise in a standard comparable manner such as to follow a common route, time, speed, etc. and for consistent home data collection. Further, the reader device may have a GPS or accelerometer sensors that enables it to tell patient where to go, e.g. “in 500 feet, turn left”, “slow down” or “speed up if possible” in accordance with preset rules for the exercise session.

In further embodiments, the reader device may include a means to collect catheter pressure measurements either through an inbuilt pressure sensor that connects to a tube-like (e.g. swann ganz) catheter, or through an analog/digital channel, that can be either wired or wireless. This may be used for taking reference measurements alongside the reader measurements.

Further, the reader device may include or be in communication with a device that includes a display to provide live or recorded pressure data either built in within the reader or integrated into an external display, e.g. smart phone or tablet. Additionally, the reader device may be configured to communicate with, or be built into an augmented reality (AR) system. In one example, the patient may wear AR glasses while exercising and the system can monitor reader output, or get instructions from the reader device on the exercise routine (e.g. ‘speed up’, ‘slow down’, ‘turn left here’, etc).

The data taken by the reader device from the implant while placed in continual communication with the implant can take advantage of various data processing steps, using measured data from the implant by itself, or in combination with other data, past or present, from the reader or from other sources. Specific methods and combinations may include comparing the ratio of PAP from the reader with cardiac output (CO) data obtained by ultrasound or some other means such as by analysis of the PAP waveform. The change in PAP/CO between rest and exercise may provide useful information to assist a clinician with diagnosing heart conditions such as pulmonary hypertension (PH), pulmonary arterial hypertension (PAH), hear failure with reduced ejection fraction (HFrEF), heart failure with preserved ejection fraction (HFpEF) as well as heart valve conditions. Further, the reader device can be used to measure, record and track a patient's diastolic PAP during sleep. This data can be useful for identifying the lowest diastolic PAP point of the day and can be a surrogate for left ventricular (LV) filling pressure or central venous pressure. This information may be useful for HF and PH stratification.

In another embodiment, a second sensor or implant may be placed in the central venous system while the other implant remains in the PA. The CV and PA measurements can be compared to estimate stroke volume and also to check tricuspid valve function, regurgitation, and RV contractility. Further still, the measured data can be combined with arterial pressure to evaluate left heart function, possibly by implanting one sensor in the PA plus a second sensor (implanted or external) on the arterial side. Systolic PA pressure may be measured to compare patient exercise and patient rest states in which this difference can be an indicator of PA stenosis, Right Heart (RH) failure, or HFrEF. PA compliance or pulmonary vascular resistance (PVR) may be derived from PA pressure change between rest and exercise, or between seated vs supine vs other positions.

Additionally, this system and method allows PAP (or other parameter) to be measured when using any exercise equipment outside of a sterile environment. Use of the system at a local physical therapy clinic, at home, or at a local gym may reduce cost and provide easier patient access than current clinic-based methods.

The system can be programmed to take measurements before/during/and after a standard six minute walk test. It can perform on-demand measurement of symptomatic arrhythmia (from PAP, ECG or other implanted sensor) at any time or place the patient feels symptoms. The system and method may be configured to take PAP or CVP measurements to determine variations in such measurement between different body positions of the patient. Such different positions include: seated, supine, prone, lateral decubitus, legs raised, head lower than torso, etc, or during valsalva maneuvers. Such measurements may be of value when assessing a congested or edemic patient, as different body positions may move fluid to different areas and can provide insight as to etiology. Further, measuring PA pressure in different positions may act as a form of spirometry. The system may also be used to estimate vessel compliance/stenosis by comparing pressures when vessels are hypo-, hyper-, or euvolemic.

The reader device may be programed to identify coughing events (short, large P spike) or dyspnea and record and report on frequency and nature of coughs or dyspnea. The system may carry out a ‘watchdog’ function: constantly or frequently measuring PAP or other parameters over long periods of time but only recording preselected events of interest. The system may include programmed algorithms, with adjustable thresholds, to determine data and processed data of clinical interest. Further, the system may measure pressure or other parameters during a surgical procedure and provide live and/or recorded pressures throughout the procedure. Measured and processed information may (i) be combined with activity monitoring sensors and algorithms to detect and classify activities and correlate activities with hemodynamic parameters; (ii) calculate changes in hemodynamic parameters between different activities and/or between rest and activities; and (iii) calculate dynamic and static arterial compliance.

The reader device may be used in a variety of environments, including but not limited to: cardiology clinics, hospitals, home, outdoor exercise, tracks, fitness center, walking route, non-cardiology clinics, (i.e., dialysis, chemo/infusion, hospital, nursing home, etc.) while at work, travel, doing errands, right before or right after taking certain medications, or at a physical therapy facility. The user may keep the reader in a purse, backpack or in a car and take ad hoc reading any time. Further, the reader may be combined with a patient management data system (such as the Endotronix Cordella PMP) to track all of the embodiments discussed above. These activity and hemodynamic data may also be combined with other patient data (e.g. clinical, physiologic, demographic, etc) obtained from other systems, devices, or databases for more accurate diagnosis, prognosis, or recommended therapy.

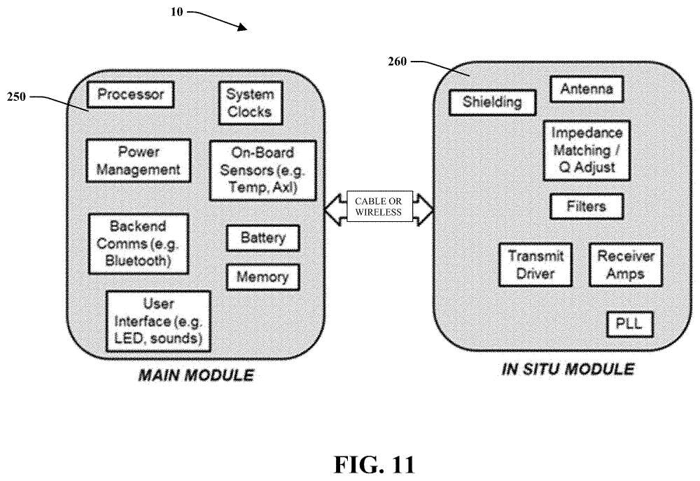

FIG. 11 illustrates a schematic diagram of another embodiment of the hemodynamic monitoring system that includes a main module 250 and an in situ module 260. Here the reader device 10 as disclosed above is generally split into these modules to reduce the physical size and weight of the reader device that must be kept against the chest of the user. It generally separates a small, light antenna/transceiver portion (in situ module) from the bulkier other subsystems (main module) of the reader device.

As such, the in-situ module 260 is configured to be placed against the chest of a patient in proximity to the target area 50 to take readings from the implant 12. The in-situ module 260 would include the antenna and may also include other electrical components such as shielding, impedance matching/Q adjusting networks, filters, transmission drivers, receiver amplifiers, or a phase locked loop. Notably, these components may also be placed in the main module so long as the antenna is able to function to send a pulse signal to the implant and receive a ring signal therefrom. The main module 250 may be in wired or wireless electrical communication with the in-situ module 260. It may include the battery of the reader device 10 as well as one or more components including a processor, power management, system clocks, on-board sensors (e.g., temperature, accelerometers), backend communication circuitry (e.g., Bluetooth), memory, and a user interface (e.g., LED, sounds, haptic feedback). The main module may be in a separate housing from the in-situ module to allow the housing of the in-situ module to be compact and thin. In one embodiment the in-situ module may be as thin as a credit card, may be mounted to a flexible substrate, and may be attached to the chest of a user by adhesives or built into a garment such as a tight-fitting shirt. The in-situ module may be a flexible circuit that can drape over a chair back, rest on a recumbent cycle, or be affixed to an exercise machine. The housing of the main module may be carried in a user's pocket, backpack, purse, or strapped to a user's belt. It may also be a separate component attachable to a user's mobile device, tablet, cell phone, or computer. Further, the in-situ module may be a flex antenna is built into tight-fitting stretchable exercise shirt that may have a connector to attach to the main module. Either module may include a thin film or flexible battery for greater space and weight saving.

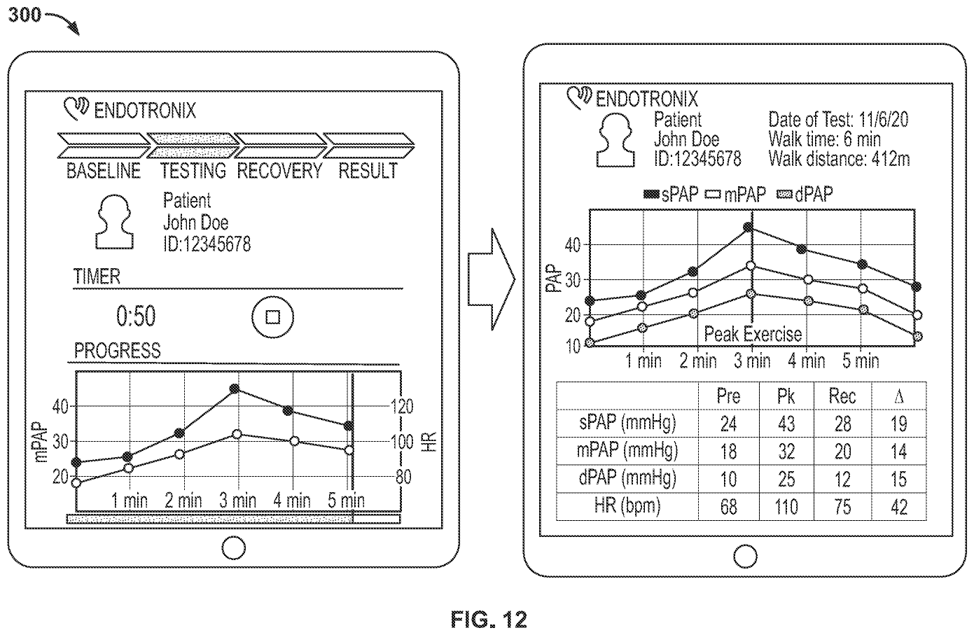

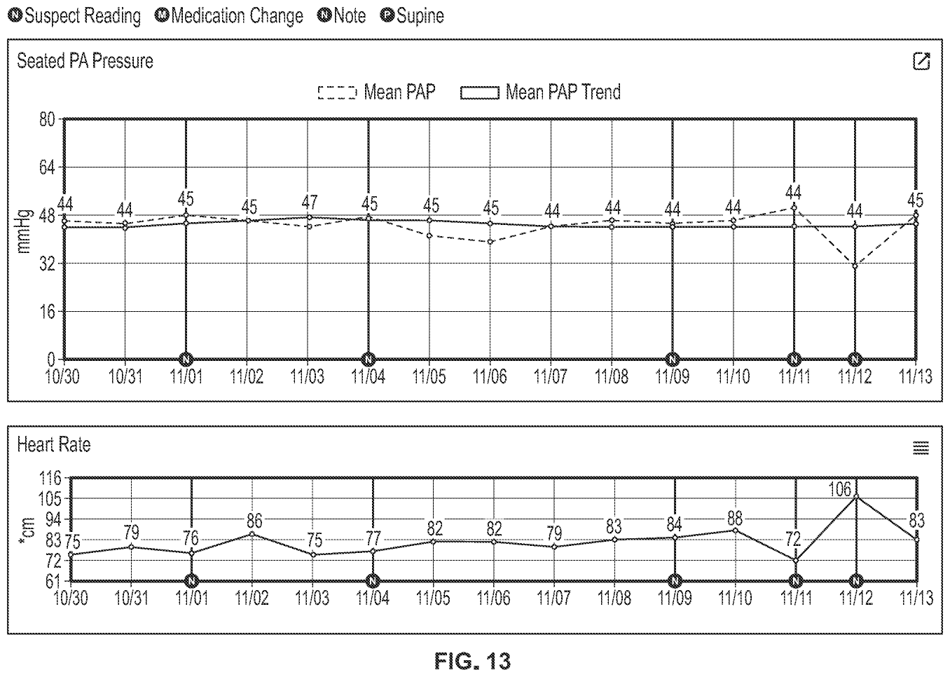

FIG. 12 illustrates screen shots of an exemplary graphical user interface 300 of a user facing display of the hemodynamic monitoring system according to the present disclosure. The tracked information in this screen includes systolic PAP (sPAP), mean PAP (mPAP), diastolic PAP (dPAP), and heart rate (HR) over a 6 minute walk. FIG. 13 illustrates a seated PA pressure graph and a heart rate graph indicating user data tracked by the hemodynamic monitoring system according to the present disclosure. This graph indicates a daily mean PAP measurement as well as mean PAP trend line over the course of several weeks, with the trend line representing a long term averaging of the daily data. The information tracked can be flagged to include a “suspect reading” when the system detects possible inaccuracy or failed reading, when a medication change occurs, a note inserted by the user (patient or clinician), and if the reading reflects a “supine” rather than seated position of the user.

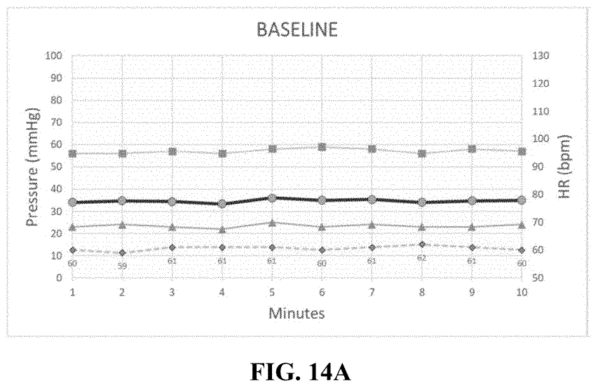

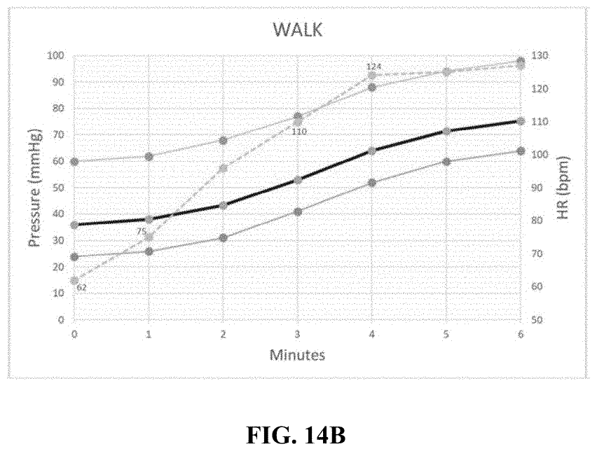

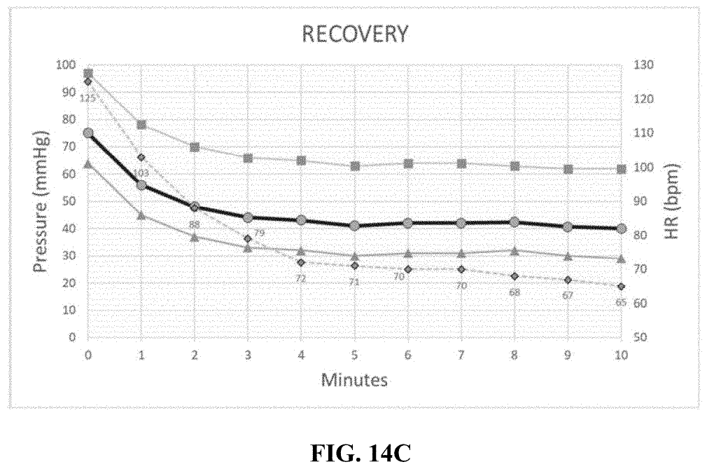

FIGS. 14A, 14B, and 14C illustrates a “baseline” pressure graph, a “walk” pressure graph, and a “recovery” pressure graph as tracked by the hemodynamic monitoring system according to the present disclosure. The tracked information in these graphs includes systolic PAP, mean PAP, diastolic PAP, and heart rate over a 10 minute baseline (at rest) duration, a 6 minute walk duration, and a 10 minute recovery (after exercise) duration. This information can be communicated to the clinician or otherwise processed through the system and displayed on a graphical user interface 300 that is user or clinician facing.

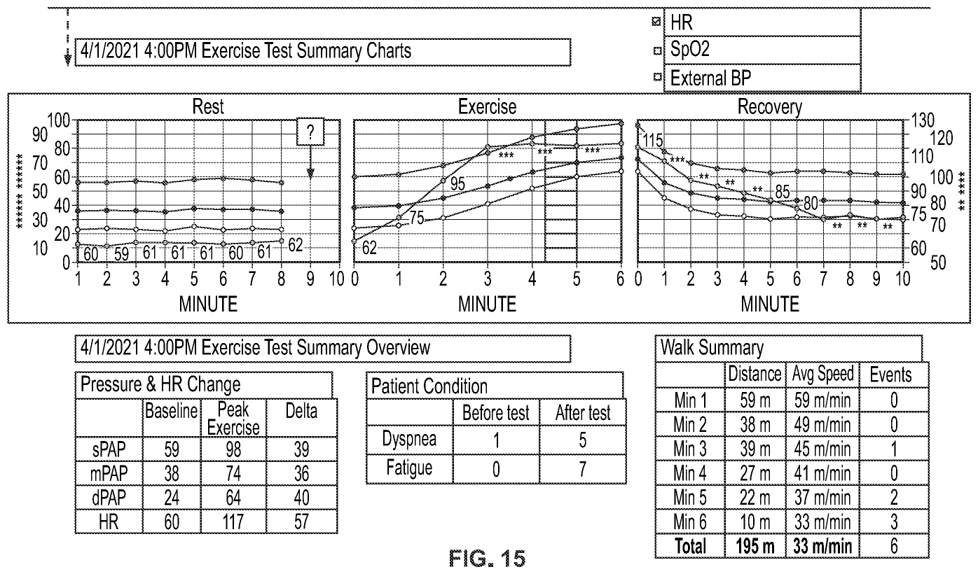

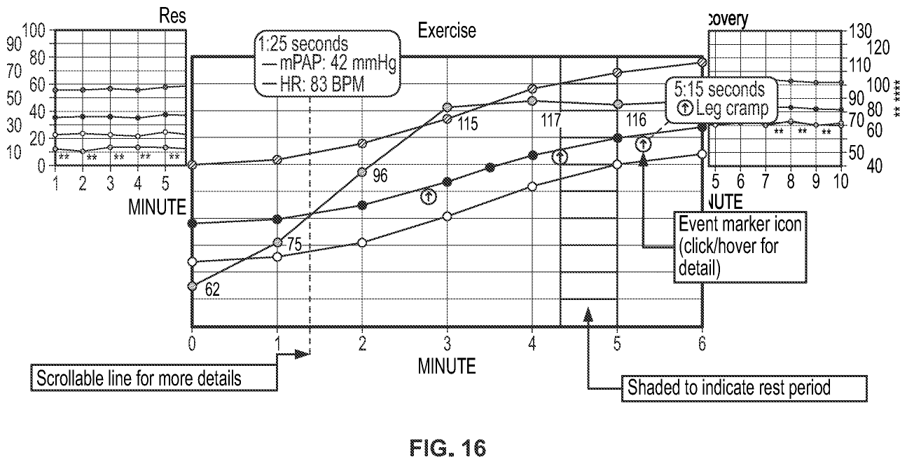

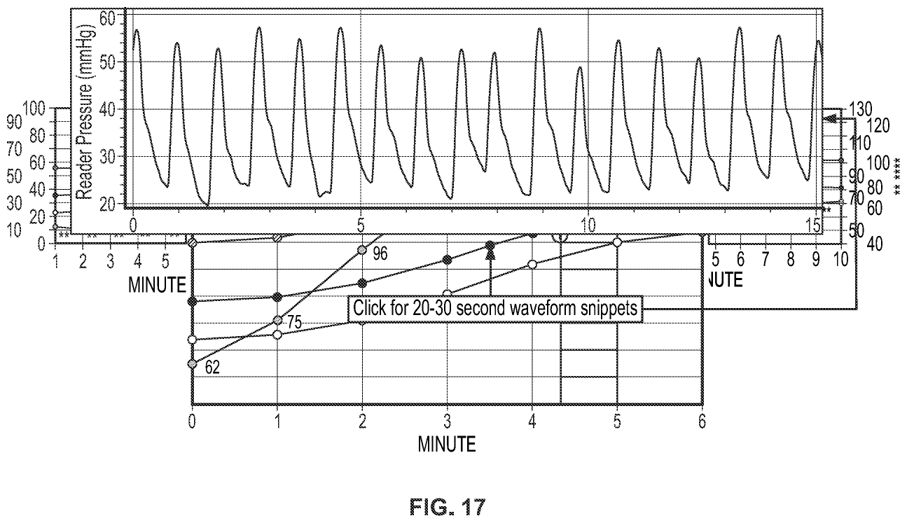

Similarly, FIGS. 15, 16, and 17 are graphs that illustrates various data readings tracked by the hemodynamic monitoring system. FIG. 15 illustrates a “rest,” “exercise,” and “recovery” graphs that could be toggled to display heart rate, SpO2, or external BP measurement as well as the change in pressure and hear rate, a patient condition, and a summary of the walk or exercise. FIG. 16 illustrates the display of an interactive graph which a user can scroll along the displayed graphical images to identify measurements along the time axis as wells as event markers that may be flagged to have occurred during the exercise. Such event markers may be considered a “leg cramp” or other such event. FIG. 17 illustrates a waveform graph window expanded from the interactive graph of FIG. 16. The graphics in FIGS. 15-17 may be used for a clinician displays, as they provide a pressure waveform, as well as greater detail regarding the hemodynamic parameters measured.

In addition to the parameters exemplified in FIGS. 12-17, the reader or an upstream processor may process past or current acquired data, by itself or in combination with other data acquired by other devices. Parameters derived by data processing may include: pressure rise & fall times and rates; location of dichrotic or anachrotic notches; area under curve; heart rate; breathing rate; pressure change due to breathing; lung capacity; cardiac output; cardiac index; stroke volume; stroke volume index; total pulmonary resistance; system and vascular pulmonary resistance; arterial compliance; heart recovery rate; heart rate variability; diastolic decay of the PA pressure; dP/dt during systolic rise; reaction of measured parameters to medicinal and other therapies. Algorithms for deriving these parameters from measured parameters such as PA pressure, alone or in combination with other measured parameters, are well documented in the art.

The embodiments disclosed here generally relate to pressure in the pulmonary artery, but similar benefits may be attained by measuring other body pressures in various patient states. These may include: Central Venous Pressure (CVP) or Right Atrial Pressure (RAP); Left Atrial Pressure (LAP); Ventricular Pressure (either side); Hepatic pressure, including pressures in the Portal Vein or in Portal shunts; Intraabdominal pressure (IAP) and pressures throughout the Gastrointestinal (GI) tract; pressures in the kidneys, ureter, and bladder; pulmonary pressures, including the lungs and the interpleural space; Intracranial Pressure (ICP); Intraocular Pressure (TOP).

In other embodiments, the concept can be further extended to other implanted sensor types including: strain sensors for orthopedics and prosthetics; electrocardiogram devices; electroencephalogram devices; implanted optical and IR cameras; chemical sensors; glucometers; blood oxidation sensors; and other sensor types well known in the art.

In other embodiments, the parameters monitored or derived may be other than hemodynamic parameters. In further embodiments, the implanted sensors may be pre-implanted into organs that are to be transplanted into patients. In still further embodiments, the implanted sensors may be built into other implanted devices, including for example shunts, stents, IVC filters, and artificial valves.

Stated further, the hemodynamic monitoring system and method may include the following steps: implanting a patient with a wireless sensing device that measures at least one hemodynamic parameter, such as pulmonary artery pressure; providing the patient with a portable external reader device that can be worn by the patient and configured to wireless communicate with said sensing device when the patient is in a specific patient state, such as resting, exercising, recovering, seated, or supine; operating the reader to take measurements from the implant when the patient is in the specific patient state, in order to acquire data from said sensing device related to said at least one parameter and uploading said data to an external device. The implant may be placed in the cardiovascular system and the measured parameters may be hemodynamic parameters. The activity information of the patient may be acquired from activity monitoring sensors and associated algorithms. A clinician may be provided with a platform to prescribe/modify exercise protocols to use frequent exercise-based measurements for treatment decisions. The clinician may also be provided with a platform to monitor live patient data during exercise testing. The reader device may also include or be placed in communication with another electronic or medical device that monitors activity (e.g. accelerometer, heartrate, GPS, etc) to signal when to take a reading or to stop taking readings. In an alternate embodiment, the implant may be actively powered (e.g. by a micro-battery). In such an embodiment it may communicate with a reader at least several meters away.

In another embodiment, provided is a system comprising a wireless implantable sensor that measures a physiological parameter and a wireless external reader that communicates with said implanted sensor. The reader has a small, portable form factor and is battery powered. A wearable harness for the reader that is configured to securely position the reader to the implanted patient's body such that its position relative to the sensor will be maintained during different patient states and the operation of reader may be hands-free. This could include the reader responding to patient's verbal commands such as “start reading”, “stop reading” etc. The implantable sensor may be placed within a cardiovascular system and configured to measure hemodynamic parameters. The wearable configuration may securely position the external reader to the patient in proximity to the implantable sensor but is also be adjustable to fit a range of body sizes and adapt to a range of optimal reading locations. In one embodiment, the reader or an upstream device includes an exercise mode that takes readings at defined intervals or is linked to an external activity monitoring device to take a reading during the patient's performance of an exercise. Such readings may be triggered by on-board reader sensors such as accelerometer, indicating start/stop of exercise. In another embodiment, the reader or an upstream device includes a sleep mode that takes readings at defined intervals or is configured to take continuous readings of the implant in the patient during sleep, or long term applications.

Notably, this method and system may be adopted for existing implant devices that are configured to provide various ongoing chronic care management services where a permanent (or long-term) implant communicates power or data with an external device in an out-patient setting or in-patient setting. The embodiments of the disclosure have been described above and, obviously, modifications and alternations will occur to others upon reading and understanding this specification. The claims as follows are intended to include all modifications and alterations insofar as they are within the scope of the claims or the equivalent thereof.

1. A hemodynamic monitoring system for wirelessly measuring a hemodynamic parameter from a remote location, comprising:
a wireless reader device configured to communicate with a wireless sensor;
said wireless sensor configured to be implanted in the vasculature of a user;
wherein the wireless reader device communicates with the wireless sensor by being placed in proximity to the wireless sensor when the wireless sensor is implanted into the body of a user to measure at least one hemodynamic parameter of the user;
wherein the wireless reader device is controlled to receive at least one response signal from said wireless sensor while the user is experiencing at least one of a set of patient states wherein the at least one response signal is representative of a physiologic parameter of the user.

2. The hemodynamic monitoring system of claim 1 wherein said hemodynamic parameter is pulmonary artery pressure recorded as a continuous waveform measured over a time period.

3. The hemodynamic monitoring system of claim 1 wherein said set of patient states is a point in time either before, during, or after at least one of: an exercise period, a sleep period, and a period when the user is experiencing symptoms or signs of a medical condition.

4. The hemodynamic monitoring system of claim 1 wherein said set of patient states is a point in time either before, during, or after at least one of: a period when the user is in a seated body posture, a period when the user is in a supine body posture, a period when the patient is in a decubitus body posture, a period when the patient is in a prone body posture, a period when the user is in a standing body posture, a period when the user is incapacitated physically or mentally, and a period when the user receives medication or other therapy.

5. The hemodynamic monitoring system of claim 1, wherein said system includes a processor configured to process said response signal to derive processed data related to hemodynamic parameters of the user acquired while the user is experiencing at least one of the set of patient states and wherein said reader is configured to communicate the response signal or the processed data to at least one processor device.

6. The hemodynamic monitoring system of claim 5 wherein said measured hemodynamic parameters includes at least one of systolic pressure, diastolic pressure, and mean pressure, pressure waveform rise or fall times, pressure waveform dichrotic or anachrotic notch characteristics, pressure waveform area under a curve, cardiac output estimation, heart rate, respiration rate, pressure change due to respiration, minimum and maximum instantaneous pressure change with time, pressure rate of change with time during inspiration or expiration, stroke volume, cardiac index, stroke volume index, total pulmonary resistance, systemic and vascular pulmonary resistance, arterial compliance, heart rate recovery, heart rate variability, and diastolic decay of pressure.

7. The hemodynamic monitoring system of claim 1 wherein said wireless reader device further comprises at least one activity monitoring sensor wherein said activity monitoring sensor is selected from among: an accelerometer; a tilt sensor; a geo-locational device; an audio, visual, haptic, touchscreen or pushbutton user interface; a heartrate sensor; a respiration rate sensor; and a body temperature sensor.

8. The hemodynamic monitoring system of claim 7 wherein data from said at least one activity monitoring sensor is used to control the wireless reader device to receive at least one response signal from said wireless sensor.

9. The hemodynamic monitoring system of claim 1 further comprising a harness device to securely position said wireless reader device to a target location on the user in proximity to the wireless sensor such that the harness device allows the user to operate said system in a hands-free manner.

10. The hemodynamic monitoring system of claim 9 wherein said harness device is at least one of: a harness; a garment; a sash; a holster; a girdle; a shirt; an adhesive strip.

11. The hemodynamic monitoring system of claim 1 wherein said external reader comprises a plurality of devices in communication with one another, at least one device located in proximity to said implanted sensor, and at least one device located away from said implanted sensor.

12. The hemodynamic monitoring system of claim 11 wherein said communication between said plurality of devices is accomplished by wired or wireless communication.

13. A method for monitoring hemodynamic parameters of a user with a wireless sensor located within the body of a user, said method comprising the steps of:
providing a reader device configured to wirelessly transfer energy, data, or commands to and from said wireless sensor;
placing the reader device in proximity to said wireless sensor at a target location on the body of the user to measure at least one physiological parameter of the user;
controlling said reader device to receive at least one response signal from said wireless sensor while the user is experiencing at least one of a set of patient states wherein the at least one response signal is representative of a hemodynamic parameter of the user.

14. The method of claim 13 wherein said set of patient states is a point in time either before, during or after at least one of: an exercise period, a sleep period, and a period when the user is experiencing symptoms or signs of a medical condition.

15. The method of claim 13 wherein said set of patient states is a point in time either before, during, or after at least one of: a period when the patient is in a seated body posture, a period when the patient is in a supine body posture, a period when the patient is in a decubitus body posture, a period when the patient is in a prone body posture, a period when the patient is in a standing body posture, a period when the patient is incapacitated physically or mentally, and a period when the patient receives medication or other therapy.

16. The method of claim 13 further comprising the step of processing said response signal to derive processed data related to physiological parameters of the user acquired while the user is experiencing at least one of the set of patient states and communicating the response signal or the processed data to at least one processor device.

17. The method of claim 13 further comprising the step of controlling the reader device from a signal received from an activity monitoring sensor, wherein the activity monitoring sensor is selected from among: an accelerometer; a tilt sensor; a geo-locational device; an audio, visual, haptic, touchscreen or pushbutton user interface; a heartrate sensor; a respiration rate sensor; and a body temperature sensor.

18. The method of claim 1 further comprising the step of providing a harness device to securely position said reader device to the target location on the user in proximity to the wireless sensor such that the harness device allows the user to operate said reader device in a hands-free manner.

19. A harness device comprising:
a front panel configured to support a reader device along a target area of a user;
a shoulder portion configured to be placed over a shoulder of a user and attached to said front panel, and
a rear portion that extends from said shoulder portion;
a support window placed in or on said front panel for supporting said reader device, the support window having a complimentary shape to a housing of said reader device wherein the housing of the reader is configured to be placed within the support window to position an antenna of the reader device against the target area and align the antenna with an implant location to allow said reader device to communicate with said implant.

20. The harness device of claim 19, further comprising at least one strap selectively attachable between the rear portion and the front panel wherein the harness device is adjustable to position and support the reader device against the target area.
